(19) United States
(12) Patent Application Publication
(10) Pub. No.: US 2014/0171645 A1
(43) Pub. Date: Jun. 19, 2014

(54) ELECTROPHILIC ALKYLATING REAGENTS, THEIR PREPARATION AND USE

(71) Applicant: Emilia Perpetua Tavares Leitao, S. Marcos (PT)

(72) Inventor: Emilia Perpetua Tavares Leitao, S. Marcos (PT)

(73) Assignee: Hovione Inter Ltd., Lucerne 7 (CH)

(21) Appl. No.: 14/006,942

(22) PCT Filed: Feb. 23, 2012
(86) PCT No.: PCT/GB2012/000189

§ 371 Date: Dec. 3, 2013

(30) Foreign Application Priority Data
Mar. 28, 2011 (PT) PT105584

(51) Int. Cl. C07J 3/00; C07C 315/02; C07C 317/14; C07D 491/18; C07C 381/12

(52) U.S. Cl. CPC C07J 3/005; C07D 491/18; C07C 381/12; C07C 317/14; C07C 315/02
USPC 546/91; 552/610; 558/44; 568/27

(57) ABSTRACT

The present invention provides electrophilic alkylating reagents of formula II, wherein is an aryl group, R₂ is an alkyl group, R₃ is a substituted phenyl group, wherein the number of substituents (n) is greater than 2 and R₄ is an anion, and salts thereof, methods for their preparation and methods for the preparation of alkylated biologically active compounds using such reagents.

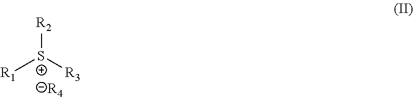

### BACKGROUND TO THE INVENTION

Alkylation is the transfer of an alkyl group from one molecule to another. The alkyl group may be transferred as an alkyl carbocation, a free radical, a carbanion or a carbene (or their equivalents). Alkylating agents are widely used in chemistry because the alkyl group is probably the most common group encountered in organic molecules. Many biologically active target molecules, or their synthetic precursors, contain one or more specific functional groups with a given order of reactivity. Selective alkylation, or addition to a desired functional group, is used, especially if there is no commonly available precursor already containing the alkyl group. Alkylating agents are classified according to their nucleophilic or electrophilic character [(a) Jerry March, 1985, Advanced Organic Chemistry reactions, mechanisms and structure, 3rd ed; (b) Stefanidakis, G.; Gwyn, J. E. 1993, “Alkylation”, In John J. McKetta. Chemical Processing Handbook)].

The classic electrophilic alkylating reagents come with a high risk of toxicity (Bolt, H. M.; Gansewendt, B. Crit. Rev; Toxicol. 1993, 23, 237-253). Methyl iodide (and others alkyl halides) are carcinogenic [(a) Pokier, L. A.; Stoner, G. D.; Shimkin, M. B. Cancer Res. 1975, 35, 1411. (b) Mc Cann, J.; Choi, E.; Yamasaki, E.; Ames, B. N. Proc. Nati. Acad. Sci. U.S.A. 1975, 72, 5135]. Dimethyl sulfate is especially dangerous, is extremely hazardous liquid and its vapor causes burns to lungs and tissues, and may be fatal if inhaled. Dimethyl carbonate has been shown to be environmental friendly [(a) Memoli S, Selva M, Tundo P. Chemosphere 2001, 43, 115. (b) Tundo, P.; Selva, M. Acc. Chem. Res. 2002, 35, 706-716]. In the presence of potassium carbonate and DBU it is especially reactive, one use being to alkylate phenols [Shieh, W.-C.; Dell, S.; Repic, O. J. Org. Chem. 2002, 67, 2188] but in the presence of a zeolite of the NaY Faujasite structure, can be chemoselective for carboxylic acids. Tetramethylammonium salts are another type of alkylating agents, considered to be non-carcinogenic and non-volatile, but with very low reactivity. Other types of alkylating reagents that can be used in mild, neutral conditions are diazo compounds such as diazomethane and trimethylsilyldiazomethane (TMSD).

Methylation is the most common type of alkylation, being associated with the transfer of a methyl group. Bromomethane (also called methyl bromide) is a favored, reagent used in methylations to prepare tertiary amines or quaternary ammonium salts present in the structure of biologically active compounds such as, but not limited to: demecarium bromide, fenpiverinium bromide, fentonium bromide, heteronium bromide, mepenzolate bromide, tropenziline bromide, vecuronium bromide, propantheline bromide, timepedium bromide, pancuronium bromide, penthienate bromide, pipecuronium bromide, pipenzolate bromide, tiotropium bromide, anisotropine methylbromide, hexafluorenium bromide, ipratropium bromide, 8-p-phenylbenzyltropinium bromide, oxitefonium bromide, and others not limited to bromide. It can also be used in the manufacture of steroids, such as: (10R,13S,17R)-methyl 11,17-dihydroxy-10,13-dimethyl-3-oxo-6,7,8,9,10,11,12,13,14,15,16,17-dodecahydro-3H-cyclopenta[a]phenanthrene-17-carboxylate.

Bromomethane is a volatile compound with a high mixing rate in the atmosphere. Bromomethane escapes easily into the atmosphere where it contributes to the depletion of ozone. It works similarly to CFCs in its reaction with ozone molecules (O₃). In the stratosphere UV light from the Sun catalyses the break down of methyl bromide forming elemental bromine which reacts with ozone, thus depleting the ozone layer. Bromomethane is 60 times more destructive to stratospheric ozone than chlorine. As such, it is subject to phase-out requirements of the 1987 Montreal Protocol on Ozone Depleting Substances (ODS).

The London Amendment in 1990 added bromomethane to the list of ODS to be phased out. In 2003 the Global Environment Facility approved funds for a UNEP-UNDP joint project for methyl bromide total sector phase out in seven countries in Central Europe and Central Asia, which was due to be completed in 2007 ((a) Merck Index, 11^{th} Edition, 5951; (b) http://www.uneptie.org/ozonaction/information/mmc/lib_detail.a sp?r=5315; c) Pool, R. 1989. The elusive replacements for CFCs. Science 242: 666).

The literature also refers to another methylating reagent, methyldiphenilsulfonium (Formula I) which has been isolated as a triflate salt (J. Am. Chem. Soc. 1993, 115, 5819-5820), a perclorate salt (Hinsberg; Chemische Berichte; vol. 69, 1936; p. 494), a hexafluorophosphate salt (Beak, Peter; Sulivan, Thomas A.; Journal of the American Chemical Society; vol. 104; nb. 16, 1982; p. 4450-4457) and as a tetrafluoroborate salt, the latter of which is commercially available.

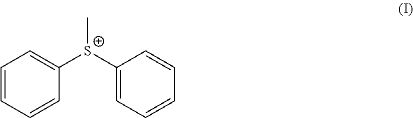

However, this compound is prepared using the methylating reagents referred to as hazards or ODS. Therefore, there is a need for alternative reagents to carry out methylations, and other alkylations in general.

We have found that stable compounds are obtained when alkyl groups are introduced into the molecule of Formula I, and to prepare these new compounds it not necessary to use the type of hazardous reagents described above. Surprisingly, these new reagents are stable and can be used to carry out alkylations.

The present invention provides new electrophilic alkylation reagents which can be converted into several salts and can successfully quaternize amines, yielding their tetrafluororate or triflate or eventually bromide or other salts, by ionic change. These reagents can also be used for the alkylation of oxygen, sulfur and phosphorous atoms. Advantageously, the compounds of the invention are solid and are not harmful to the ozone layer.

### DESCRIPTION OF THE INVENTION

This invention aims to avoid the use of reagents that deplete the ozone layer or are hazardous by offering alternative reagents to carry out alkylation reactions that are less problematic in their use.

Accordingly, in a first embodiment of the present invention, there is provided a compound of Formula II or a salt thereof,

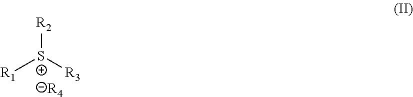

wherein:
R₁ is an aryl group;
R₂ is an alkyl group;
R₃ is a substituted phenyl group, wherein the number of substituents (n) is greater than 2; and
R₄ is an anion; preferably tetrafluoroborate, triflate, sulfate, phosphate or carbonate.

Preferably, R₁ is an optionally substituted C₆₋₁₂ aryl group and more preferably R₁ is an optionally substituted phenyl group, such as phenyl substituted by one or more C₁₋₄ alkyl groups. Most preferably, R₁ is a phenyl or p-tolyl group.

Preferably, R₂ is a linear or branched chain C₁₋₆ alkyl group, more preferably a linear or branched chain C₁₋₆ alkyl group, and most preferably a linear or branched chain C₁₋₄ alkyl group. Preferred examples of R₂ include methyl, ethyl, n-propyl, iso-propyl, n-butyl, sec-butyl, iso-butyl and tert-butyl. More preferred examples of R₂ include methyl and ethyl.

Preferably, R₃ is a phenyl group substituted by two or more alkyl groups, preferably two or more linear or branched chain C₁₋₆ alkyl groups, and more preferably two or more linear or branched chain alkyl groups. Preferred alkyl group substituents include methyl, ethyl, n-propyl, iso-propyl, n-butyl, sec-butyl, iso-butyl and tert-butyl. More preferred alkyl group substituents include methyl and ethyl, particularly methyl.

Preferably, (n) is 2, 3, 4 or 5. More preferably, (n) is 2, 3 or 4. Most preferably, (n) is 4.

Most preferably, R₄ is tetrafluoroborate, triflate or hexfluorophosphate.

In a further preferred embodiment of the invention, there is provided a subset of compounds of Formula II, of Formula III, or a salt thereof,

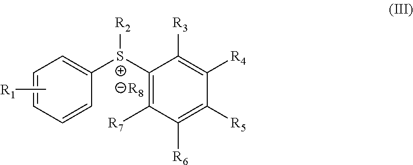

wherein:
R₁ is an alkyl group or H;
R₂ is an alkyl group;
R₃, R₄, R₅, R₆ and R₇, which may be the same or different, are each independently selected from an alkyl group or H; and
R₈ is an anion; preferably tetrafluoroborate, triflate, sulfate, phosphate or carbonate.

When R₁ is an alkyl group, it is preferably a linear or branched chain C₁₋₄ alkyl group such as methyl, ethyl, n-propyl, iso-propyl, n-butyl, sec-butyl, iso-butyl or tert-butyl, most preferably methyl. In an alternative preferred embodiment, R₁ is H.

Preferably, R₂ is a linear or branched chain C₁₋₈ alkyl group, more preferably a linear or branched chain C₁₋₆ alkyl group, and most preferably a linear or branched chain C₁₋₄ alkyl group. Preferred examples of R₂ include methyl, ethyl, n-propyl, iso-propyl, n-butyl, sec-butyl, iso-butyl and tert-butyl. More preferred examples of R₂ include methyl and ethyl.

Preferably, one or two of R₃, R₄, R₅, R₆ and R₇ are H and each of the remaining substituents, which may be the same or different, is an alkyl group, preferably linear or branched chain C₁₋₄ alkyl groups such as methyl, ethyl, n-propyl, iso-propyl, n-butyl, sec-butyl, iso-butyl or tert-butyl, particularly methyl.

In a particularly preferred embodiment, each of R₄, R₅ and R₆ which may be the same or different, is an alkyl group, preferably a linear or branched chain C₁₋₄ alkyl group, and one of R₃ and R₇ is H and the other is an alkyl group, preferably a linear or branched chain C₁₋₄ alkyl group, particularly methyl.

In a further preferred embodiment, each of R₃, R₄, R₅ and R₆ is an alkyl group, preferably a linear or branched chain C₁₋₄ alkyl group, particularly methyl, and R₇ is H.

Most preferably, R₈ is tetrafluoroborate, triflate or hexfluorophosphate.

Particularly preferred compounds of the present invention include:
S-methyl-S-phenyl-2,3,4,5-tetramethyl phenyl sulfonium triflate;
S-methyl-S-phenyl-2,3,4,5-tetramethyl phenyl sulfonium tetrafluoroborate;
S-methyl-S-phenyl-2,3,4,5-tetramethyl phenyl sulfonium hexafluorophosphate;
S-methyl-S-phenyl-2,3,4,5-tetramethyl phenyl sulfonium hexafluorophosphate;
methyl(2,3,4,5-tetramethylphenyl)(p-tolyl) sulfonium triflate;
methyl (2,3,4,5-tetramethylphenyl)(p-tolyl) sulfonium tetrafluoroborate;
S-ethyl-S-(4-methyl) phenyl-2,3,4,5-tetramethyl phenyl sulfonium triflate;
S-ethyl-S-phenyl-2,3,4,5-tetramethyl phenyl sulfonium tetrafluoroborate; and
S-ethyl-S-phenyl-2,3,4,5-tetramethyl phenyl sulfonium hexafluorophosphate.

Organic biologically active compounds can be obtained by the alkylation reaction between organic substrates with compounds of Formula II or preferably compounds of Formula III. By “organic biologically active compounds” we mean an organic compound which is of medical or therapeutic use in the broadest sense. Typically, the organic biologically active compounds are pharmaceutically active compounds.

The alkylating reagents of this invention, according to Formula II and Formula III, can be conveniently prepared in a one pot reaction, by isolating each intermediate or combining reactions and isolating only some of the intermediates.

Accordingly, in a further aspect of the present invention there is provided a process for preparing a compound of Formula II, or a salt thereof,

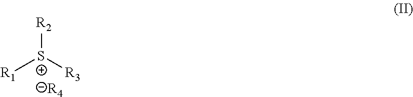

wherein
R₁ is an aryl group;
R₂ is an alkyl group;
R₃ is a substituted phenyl group, wherein the number of substituents (n) is greater than 2; and
R₄ is an anion; preferably tetrafluoroborate, triflate, sulfate, phosphate or carbonate; which process comprises (i) reacting a compound of Formula IV,

R₁—S(═O)—R₂ (IV)

wherein R₁ and R₂ are as defined in Formula II, with a suitable substituted phenyl derivative; (ii) reacting the compound so formed with a suitable anion source; and optionally thereafter (iii) converting one compound of Formula II into another.

Examples of suitable compounds of Formula IV include methyl phenyl sulfoxide and ethyl phenyl sulfoxide. Compounds of Formula IV are commercially available or may be prepared from their corresponding sulphide precursors (such as methyl- or ethyl phenyl sulphide) using conventional methods known in the art, as exemplified by the methods of Examples 1 and 7 herein.

Examples of suitable substituted phenyl derivatives include 1,2,3,4-tetramethylbenzene, 1,2,3-trimethylbenzene and 1,2,4,5-tetramethylbenzene. Such compounds are commercially available or may be prepared using conventional methods known in the art.

Examples of suitable anion sources include trifluoromethanesulfonic anhydride, sodium tetrafluoroborate and hexafluorophosphoric acid. Such compounds are commercially available or may be prepared using conventional methods known in the art.

The reactions to prepare compounds of Formulae II and III are preferably carried out starting from an organic compound of the type described in Table 1.

TABLE 1
Starting materials

| Formula | Substituent |
|---|---|
| 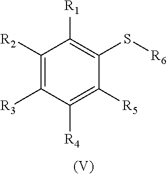 | R1, R2, R3, R4, R5 = H, alkyl, aryl, alkoxy, halogen R6 = alkyl |
| 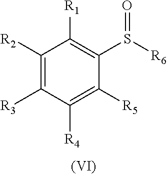 | |
| 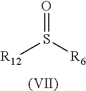 | R12 = resin, naphthalene, substituted naphthalene R6 = alkyl |

Compounds of Formula V, VI and VII are commercially available or may be prepared by conventional methods known in the art.

Accordingly, in a further aspect of the present invention there is provided a process for preparing a compound of Formula II or Formula III, which process comprises the step of preparing a compound of Formula V, VI or VII,

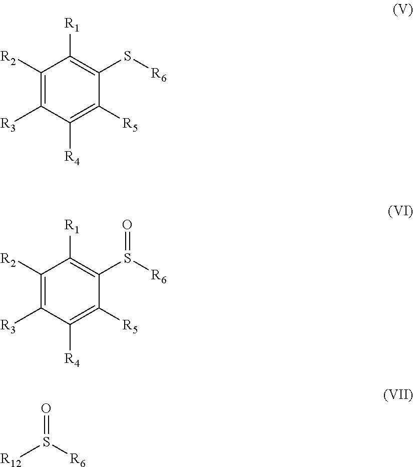

wherein,
R₁, R₂, R₃, R₄ and R₅, which may be the same or different, are independently selected from H, alkyl, aryl, alkoxy and halogen;
R₆ is an alkyl group; and
R₁₂ is H, optionally substituted naphthalene or a polymer resin.

The terms “alkyl” and “aryl” as used with reference to compounds of Formulae V, VI and VII have the same meanings as hereinbefore defined in with reference to compounds of Formula II.

The term “alkoxy” refers preferably to a linear or branched chain C₁₋₈ alkoxy group, more preferably to a linear or branched chain C₁₋₆ alkoxy group, and most preferably to a linear or branched chain C₁₋₄ alkoxy group. Preferred examples of alkoxy groups include methoxy, ethoxy, propoxy and butoxy.

The term “halogen” means F, Cl, Br or I, preferably Cl or Br.

When R₁₂ represents substituted naphthalene, preferred examples include 1-methylnaphthalene and 2-methylnaphthalene.

When R₁₂ represents a resin, preferred examples include (Poly(styrene-co-divinylbenzene) and other benzenic resins.

The reactions to prepare compounds of Formulae II and III can be carried at a temperature in the range between from about −70° C. to about 70° C., preferably from about −10° C. to about 25° C.

The reactions to prepare compounds of Formulae II and III are typically carried out in the presence of a suitable organic solvent. Examples of suitable solvents include, but are not limited to, diethyl ether, dichloromethane and acetonitrile.

The reaction may also be carried out in the presence of an organic or inorganic catalyst. Examples of suitable catalysts include, but are not limited to metallic catalysts, preferably transition metals and transition metal complexes.

The reactions can be carried out in the presence of other chemical compounds such as: oxidizing agents (such as oxygen peroxide), reductive agents (such as hydrogen), organic or inorganic bases and/or halogenating agents (such as thionyl chloride and phosphorus pentachloride). The reagents can also be prepared as salts.

The compounds of Formulae II and III can be isolated by direct crystallization from the reaction mixture; by the addition of an anti-solvent which can be an organic solvent or water, or water with acid, or water with base (or by the addition of water, or water with acid, or water with base to the reaction mixture); by extraction with organic solvent and/or concentration (organic layers can be washed with water, and/or bases, and or acids, and or reductive agents and/or can be dried using a drying agents); by recrystallization in organic solvent; and/or by column chromatography. Resins and/or activated charcoal can also be used during the work-up to purify the alkylating reagent end-product.

The starting materials can be used either as described above (i.e. in “free form”) or may be bound to a suitable solid phase support, such as a resin. When the reagents are bound to a solid phase support this typically facilitates the removal of by-products from the reaction mixture.

One aspect of this invention concerns methods for preparation of alkylated organic biologically active compounds using the alkylating reagents described above and/or and salts thereof.

Thus, in one embodiment the present invention provides the use of a compound of Formula II or Formula III, or a salt thereof, as an alkylating agent, preferably for the alkylation of an organic biologically active compound, such as a pharmaceutically active compound.

In an alternative embodiment, there is provided the use of a compound of Formula II or Formula III, or a salt thereof, for the preparation of an organic biologically active compound, such as a pharmaceutically active compound.

In a further alternative embodiment, there is provided a method of manufacture of an organic biologically active compound comprising at least one alkylation step, wherein the alkylating reagent is a compound of Formula II or a salt thereof.

The alkylated organic biologically active compounds can be prepared by reaction of the alkylating reagents (of Formulae II and III) with a suitable substrate. Examples of suitable substrates include, but are not limited to, N-Demethyltiotropium [(Scopine di(2-thienyl)glycolate] or (10R,13S,17R)-11,17-dihydroxy-10,13-dimethyl-3-oxo-6,7,8,9,10,11,12,13,14,15,16,17-dodecahydro-3H-cyclopenta[a]phenanthrene-17-carboxylic acid

The reactions to prepare alkylated organic biologically active compounds can be carried at a temperature in the range between from about −70° C. to about 70° C., preferably from about −10° C. to about 25° C.

Such reactions are typically carried out in the presence of a suitable organic solvent. Examples of suitable solvents include, but are not limited to, acetonitrile, heptane, hexane, cyclohexane, methyl tert-butyl ether (MTBE), dimethylformamide (DMF), toluene, 1,2-dichloromethane, α,α,α-trifluorotoluene, tetrahydrofurane (THF), methyl-THF, 1,2-dimethoxyethane and mixtures thereof.

The reactions can also be carried out in the presence of an organic or inorganic catalyst such as preferably transition metals and transition metal complexes.

The reactions can also be carried out in the presence of other chemical compounds such as: oxidizing agents (such as oxygen peroxide), reductive agents (such as hydrogen), organic or inorganic bases (such as a weak base, in particular caesium carbonate, sodium carbonate, potassium carbonate and/or mixtures thereof), and/or halogenating agents (such as thionyl chloride and Phosphorus pentachloride).

The alkylated organic biologically active compounds obtained can be isolated by crystallization from the reaction mixture or by addition of an anti-solvent to the reaction mixture. The anti-solvent can be an organic solvent or water, water with acid or water with base. The alkylated organic biologically active compounds can also be isolated by the addition of water to the reaction mixture (or water with acid, or water with base). They can also be isolated by extraction with an organic solvent and/or concentration. When the alkylated organic biologically active compounds are isolated by extraction, the organic layers can be washed with water and/or base, and or acid, and or reductive agent solution. After or during the washes the organic layers can be dried with drying agents. Resin and activated charcoal can also be used during the work-up to purify the organic extracts. The alkylated organic biologically active compounds isolated from the reactions as described above can be purified by recrystallization or by column chromatography and can be isolated as a dry power by spray drying or lyophilisation.

Examples of alkylated organic biologically active compounds that may be prepared using the alkylating reagents of Formulae II and III include, but are not limited to: demecarium bromide, fenpiverinium bromide, fentonium bromide, heteronium bromide, mepenzolate bromide, tropenziline bromide, vecuronium bromide, propantheline bromide, timepedium bromide, pancuronium bromide, penthienate bromide, pipecuronium bromide, pipenzolate bromide, tiotropium bromide, anisotropine methylbromide, hexafluorenium bromide, ipratropium bromide, 8-p-phenylbenzyltropinium bromide, oxitefonium bromide, and others.

Preferably, the compounds to be prepared using alkylating reagents of Formulae II and III include: tiotropium bromide, anisotropine methylbromide, fenpiverinium bromide, fentonium bromide, hexafluorenium bromide, ipratropium bromide, 8-p-phenylbenzyltropinium bromide, most preferably tiotropium bromide and ipratropium bromide.

The alkylation reaction is especially suited to lower alkylation reactions and the organic biologically active compounds obtained are for example methylated or ethylated compounds.

The alkylation reaction of this invention is not limited only to the alkylation of nitrogen atoms but is also applicable to the alkylation of compounds at other heteroatoms, such as a sulfur atom as well an oxygen atom. Examples of biologically active compounds that may be produced in this way include, but are not limited to, Etisul® (diethyldithiolisophthalate) and Stimovul® (epimestrol).

The alkylated organic biologically active compounds obtained can be used as active pharmaceutical ingredients, formulated as known and used in the treatment of several medical conditions.

The following examples are given below, merely as illustrative of the invention and are not intend to limit the scope of the invention in any way.

The products obtained in these examples were characterized/analysed using the following apparatus:
¹H NMR spectra were recorded on a Bruker Avance II 400 MHz spectrometer.
IR spectra were recorded on a Mattson Research Series FTIR spectrometer.
The melting points were measured in a Buchi Melting Point B-540.
HPLC were recorded in a Water 600 controller; injector waters 717 plus autosampler; detector waters 996 (PAD).
DSC plots were measured in a TA Instruments, DSC Q200
MS were recorded in a LC-Waters Alliance 2690 with a PDA detector Waters 996; MS: Micromass, Quattro LC or in a LC-Waters Alliance 2695 with a PDA detector Waters 2996; MS: Micromass, Quattro micro

### EXAMPLES

### Example 1

### Preparation of Methyl Phenyl Sulfoxide

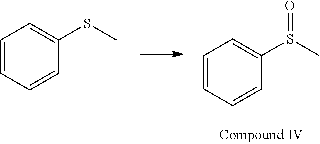

Methyl phenyl sulfide (18.9 mL, 159.42 mmol) was dissolved in dichloromethane (120 mL) and the solution was cooled to a temperature between 5° C. and 0° C. A mixture of methanol (200 mL) and water (20 mL) was added to the previous solution. NBS (28.7 g, 1.01 eq) was added in small portions, maintaining the same temperature range. The reaction mixture was stirred within the same temperature range until the reaction was complete. Thereafter, the reaction mixture was quenched by the addition of Na₂SO₃ solution (10%, 150 mL). The pH of the reaction mixture was adjusted to a value between 7 and 8, with saturated NaHCO₃ solution. The resulting mixture was extracted with dichloromethane (2×100 mL). The combined organic layer was dried with anhydrous Na₂SO₄ and concentrated. Heptane (30 mL) was added and the solution was concentrated again. 19.7 g of the desired product was obtained as a colourless liquid.

### Example 2

### Preparation of S-methyl-S-phenyl-2,3,4,5-tetramethyl phenyl sulfonium triflate

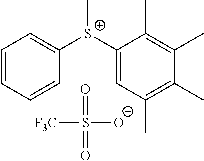 [SMILES: Cc1cc([S+](C)c2ccccc2)c(C)c(C)c1C ; O=S(=O)([O-])C(F)(F)F]

Methyl phenyl sulfoxide (9.45 g, 67.40 mmol) was dissolved in dry diethyl ether (50 ml) under nitrogen atmosphere. 1,2,3,4-Tetramethylbenzene (10.1 mL, 1.0 eq) was added to the previous solution and then the mixture was cooled to a temperature between 5° C. and 0° C. After stabilizing the temperature, trifluoromethanesulfonic anhydride (11.36 mL, 1.0 eq) was added slowly, maintaining the same temperature. The mixture was stirred until the reaction was complete. The precipitated triflate salt was isolated by filtration, washed with diethyl ether at 0° C. dried. 22.26 g of the desired product was obtained as a white solid (Yield: 93.67%); m.p.: 136-137° C.; ¹H NMR (solvent CDCl₃, 400 MHz): δ 7.78-7.77 (2H, m), 7.65-7.59 (3H, m), 7.50 (1H, s), 3.61 (3H, s); 2.48 (3H, s), 2.39 (3H, s), 2.27 (3H, s), 2.26 (3H, s). ¹³C NMR (CDCl₃, 100 MHz): δ 143.1, 138.8, 138.5, 136.1, 133.8, 131.3, 129.4, 126.7, 126.1, 119.9, 28.5, 20.9, 17.3, 16.8, 16.7. FT-IR (KBr): 3014, 2931, 1477, 1448, 1265, 1224, 1149, 1031 cm⁻¹.

### Example 3

### Preparation of S-methyl-S-phenyl-2,3,4,5-tetramethyl phenyl sulfonium tetrafluoroborate

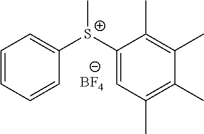

S-methyl-S-phenyl-2,3,4,5-tetramethyl phenyl sulfonium triflate (10 g) of example 1 was dissolved in dichloromethane (60 mL). The solution was washed 6 times with a sodium tetrafluoroborate aqueous solution (1N). The resulting organic layer was dried with anhydrous sodium sulfate and concentrated to dryness. 7.12 g of the desired product was obtained as a white solid. (Yield: 72.89%); m.p.: 209-211° C.; ¹H NMR (CDCl₃, 400 MHz): δ 7.76-7.74 (2H, m), 7.64-7.59 (3H, m), 7.47 (1H, s), 3.56 (3H, s), 2.47 (3H, s), 2.40 (3H, s), 2.27 (3H, s), 2.26 (3H, s). ¹³C NMR (solvent CDCl₃, 100 MHz): δ 143.1, 138.7, 138.5, 136.1, 133.8, 131.3, 129.3, 126.7, 126.0, 119.8, 28.2, 20.9, 17.2, 16.8, 16.7. FT-IR (KBr): 3043, 3023, 2942, 1583, 1477, 1448, 1384, 1288, 1220, 1172, 1051 cm⁻¹.

### Example 4

### Preparation of S-methyl-S-phenyl-2,3,4,5-tetramethyl phenyl sulfonium hexafluorophosphate

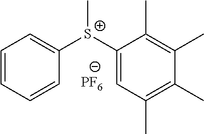

S-methyl-S-phenyl-2,3,4,5-tetramethyl phenyl sulfonium triflate (0.200 g, 0.49 mmol) of example 1 was dissolved in CH₂Cl₂ (5 mL). The solution was added to a solution of HPF₆ [prepared by diluting 0.221 mL of HPF₆ (65%, wt in H₂O, 2 eq), in 5 mL of H₂O]. The mixture was strongly stirred for 3 h, the phases were separated, the aqueous phase was extracted with CH₂Cl₂ (3×5 mL), dried with anhydrous MgSO₄ and concentrated to afford 0.198 g (100%) of the desired product as a white solid. Mp=232-234° C. ¹H NMR (DMSO-d₆, 400 MHz): d 7.95-7.93 (2H, m), 7.75-7.68 (4H, m), 3.76 (3H, s), 2.49 (3H, s), 2.35 (3H, s), 2.25 (6H, s), 2.21 (3H, s). ¹³C NMR (DMSO-d₆, 100 MHz): d 141.7, 137.9, 137.0, 135.8, 133.4, 133.0, 129.5, 127.9, 122.2, 26.5, 20.3, 17.0, 16.38, 16.32. ³¹P NMR (DMSO-d₆, 161.978 MHz): d −144.1 (hept, J=711.7 Hz). FT-IR (KBr): 3033, 2950, 1583, 1477, 1450, 1419, 1388, 995, 838 cm⁻¹.

### Example 5

### Preparation of methyl (2,3,4,5-tetramethylphenyl)(p-tolyl) sulfonium triflate

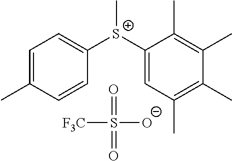

Methyl p-tolyl sulfoxide (1 g; 6.48 mmol) was dissolved in dry diethyl ether (15 mL) under nitrogen atmosphere. 1,2,3,4-Tetramethylbenzene (0.92 ml, 1 eq) was added to the previous solution and then the reaction mixture was cooled to a temperature between −5° C. and −10° C. After stabilizing the temperature, trifluoromethanesulfonic anhydride (1.3 mL, 1.2 eq) was added slowly, maintaining the same temperature. The mixture was stirred until the reaction was complete. The precipitated triflate salt was isolated by filtration, washed with diethyl ether at 0° C. and dried under vacuum. 2.09 g of the desired product was obtained as a white solid; (Yield: 76.8%); m.p.: 104-105° C.; ¹H NMR (solvent CDCl₃, 400 MHz): δ 7.67 (2H, d, J=8.3 Hz), 7.48 (1H, s), 7.40 (2H, d, J=8.3 Hz), 3.57 (3H, s), 2.47 (3H, s), 2.40 (3H, s), 2.38 (3H, s), 2.26 (3H, s), 2.25 (3H, s). ¹³C NMR (CDCl₃, 100 MHz): δ 145.3, 142.9, 138.7, 138.3, 135.8, 132.0, 129.4, 125.8, 122.9, 120.4, 28.5, 28.4, 21.5, 20.8, 17.2, 16.75, 16.73. FT-IR (KBr): 3039, 2937, 1427, 1263, 1224, 1172, 1153, 1029, 991 cm⁻¹.

### Example 6

### Preparation of methyl (2,3,4,5-tetramethylphenyl)(p-tolyl) sulfonium tetrafluoroborate

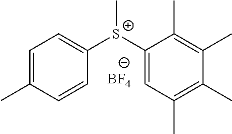

Methyl (2,3,4,5-tetramethylphenyl)(p-tolyl) sulfonium trifluoromethahesulfonate (1.2 g) of example 4, was dissolved in dichloromethane (30 ml). The solution was washed 6 times with a sodium tetrafluoroborate solution (1N). The resulting organic layer was dried with anhydrous sodium sulfate and concentrated to dryness. 0.83 g of the desired product was obtained as a white solid. (Yield: 69.1%); m.p.: 136-137° C.; m.p.: 171-172° C.; ¹H NMR (CDCl₃, 400 MHz): δ 7.68 (2H, d, J=8.4 Hz), 7.51 (1H, s), 7.40 (2H, d, J=8.3 Hz), 3.51 (3H, s), 2.46 (3H, s), 2.39 (6H, s), 2.25 (3H, s), 2.24 (3H, s). ¹³C NMR (solvent CDCl₃, 100 MHz): δ 145.1, 142.6, 138.5, 138.2, 135.7, 131.8, 129.3, 125.7, 122.9, 120.4, 27.8, 21.3, 20.6, 17.0, 16.62, 16.60. FT-IR (KBr): 3041, 3023, 2940, 1592, 1492, 1446, 1398, 1286, 1124, 1052, 970 cm⁻¹.

### Example 7

### Preparation of Ethyl Phenyl Sulfoxide

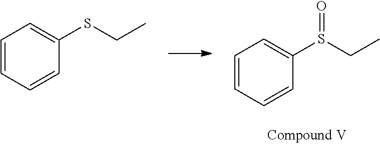

Ethyl phenyl sulfide (17.5 mL, 128.77 mmol) was dissolved in dichloromethane (333 mL) and the solution was cooled to a temperature between 5° C. and 0° C. A mixture of methanol (193 mL) and water (19.3 mL) was added to the previous solution. NBS (34.5 g, 1.5 eq) was added in small portions, maintaining the same temperature range. The reaction mixture was stirred within the same temperature range until the reaction was complete. Thereafter, the reaction mixture was quenched by the addition of Na₂SO₃ solution (10%, 500 mL). The pH of the reaction mixture was adjusted to a value between 7 and 8, with saturated NaHCO₃ solution. The resulting mixture was extracted with dichloromethane (2×100 mL). The combined organic layer was dried with anhydrous Na₂SO₄ and concentrated. Heptane (30 mL) was added and the solution was concentrated again. The crude product was purified by column chromatography (ethyl acetate/hexane 8:4) to give the desired product as a colourless liquid.

### Example 8

### Preparation of S-ethyl-S-(4-methyl) phenyl-2,3,4,5-tetramethyl phenyl sulfonium triflate

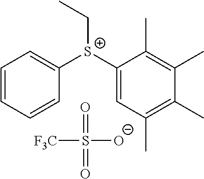 [SMILES: CC[S+](c1ccccc1)c1cc(C)c(C)c(C)c1C ; O=S(=O)([O-])C(F)(F)F]

Ethyl phenyl sulfoxide (1.3 mL, 9.21 mmol) of example 7 was dissolved in dry diethyl ether (15 ml) under nitrogen atmosphere. 1,2,3,4-Tetramethylbenzene (1.37 ml, 1.0 eq) was added. The reaction mixture was stirred at a temperature between 20° C. and 25° C. Trifluoromethanesulfonic anhydride (1.68 mL, 9.95 mmol) was added slowly, maintaining the same temperature range. The mixture was stirred, at the same temperature range, until the reaction was complete. The precipitated triflate salt was isolated by filtration, washed twice with cold diethyl ether and dried under vacuum. 2.41 g of the desired product was obtained as a white solid; (yield: 62.2%); m.p.: 105-106° C.; ¹H NMR (solvent CDCl₃, 400 MHz): δ 7.86-7.84 (2H, m), 7.67-7.62 (4H, m), 4.31-4.25 (1H, m), 4.12-4.07 (1H, m), 2.50 (3H, s), 2.41 (3H, s), 2.27 (3H, s), 2.26 (3H, s), 1.47 (3H, t, J=7.2 Hz). ¹³C NMR (CDCl₃, 100 MHz): δ 143.2, 138.8, 138.7, 136.9, 134.0, 131.3, 130.1, 126.7, 125.3, 118.6, 39.9, 20.8, 17.4, 16.84, 16.81, 9.6. FT-IR (KBr): 3073, 3014, 2992, 2948, 1479, 1448, 1388, 1259, 1222, 1147, 1033 cm⁻¹.

### Example 9

### Preparation of S-ethyl-S-phenyl-2,3,4,5-tetramethyl phenyl sulfonium tetrafluoroborate

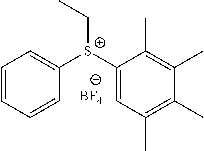

S-ethyl-S-(4-methyl) phenyl-2,3,4,5-tetramethyl phenyl sulfonium triflate (1.2 g) of example 8; was dissolved in dichloromethane (30 mL). The solution was washed 6 times with a sodium tetrafluoroborate solution (1N). The resulting organic layer was dried with sodium sulfate and concentrated to dryness. 0.83 g of the desired product was obtained as a white solid. (Yield: 68.1%); m.p.: 148-150° C.; ¹H NMR (solvent CDCl₃, 400 MHz): δ 7.84-7.82 (2H, m), 7.66-7.59 (4H, m), 4.19-4.13 (1H, m), 4.06-4.01 (1H, m), 2.50 (3H, s), 2.40 (3H, s), 2.27 (3H, s), 2.26 (3H, s), 1.47 (3H, t, J=7.2 Hz). ¹³C NMR (CDCl₃, 100 MHz): δ 143.1, 138.8, 138.6, 136.9, 134.0, 131.3, 130.1, 126.5, 125.2, 118.6, 39.7, 20.8, 17.3, 16.8, 16.7, 9.6. FT-IR (KBr): 2983, 2954, 2931, 1585, 1448, 1384, 1268, 1222, 1047 cm⁻¹.

### Example 10

### Preparation of S-ethyl-S-phenyl-2,3,4,5-tetramethyl phenyl sulfonium hexafluorophosphate

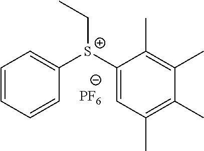

S-ethyl-S-(4-methyl) phenyl-2,3,4,5-tetramethyl phenyl sulfonium triflate (0.200 g, 0.48 mmol) of example 8, was dissolved in CH₂Cl₂ (5 mL). The solution was added to a solution of HPF₆ (prepared by diluting 0.214 mL of HPF₆ [65% wt in H₂O, 2 eq), in 5 mL of H₂O). The mixture was strongly stirred for 3 h, the phases were separated, the aqueous phase was extracted with CH₂Cl₂ (3×5 mL), dried with anhydrous MgSO₄ and concentrated to afford 0.198 g (92%) of the desired product as a white solid. Mp=172-174° C. ¹H NMR (DMSO-d₆, 400 MHz): d 8.00-7.79 (2H, m), 7.77-7.71 (4H, m), 3.76 (3H, s), 4.37-4.31 (1H, m), 4.24-4.19 (1H, m), 2.48 (3H, s), 2.36 (3H, s), 2.25 (6H, s). ¹³C NMR (DMSO-d₆, 100 MHz): d 142.1, 138.2, 137.3, 136.7, 133.9, 131.0, 130.2, 126.4, 125.8, 120.3, 38.3, 20.3, 17.0, 16.4, 16.3, 9.1. ³¹P NMR (DMSO-d₆, 161.978 MHz): d −144.1 (hept, J=711.7 Hz). FT-IR (KBr): 3031, 2989, 2956, 2929, 1585, 1477, 1452, 1425, 1386, 1218, 1079, 1024, 1002 cm⁻¹.

### Example 11

### Preparation of Tiotropium Bromide[(1α,2β,4β,7β)-7-[(hydroxidi-2-thienylacetyl)oxy]-9,9-dimethyl-3-oxa-9-azoniatricyclo(3.3.1.0″]nonane bromide]

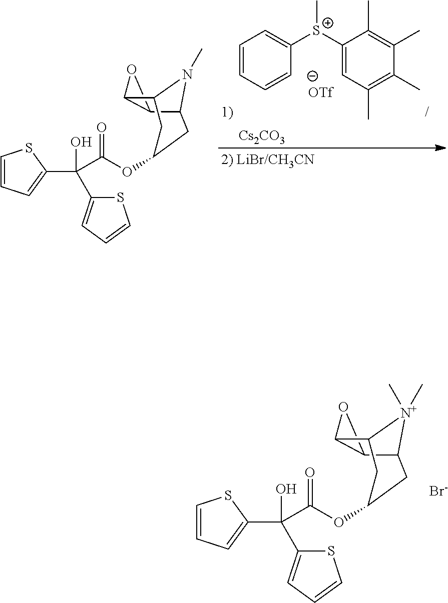

N-Demethyltiotropium[(Scopine di(2-thienyl)glycolate] (250 mg, 662.29 μmol) was dissolved in acetonitrile (5 ml) and S-methyl-S-phenyl-2,3,4,5-tetramethyl phenyl sulfonium triflate of example 2 (536.17 mg, 1.0 eq) was added. The resulting mixture was stirred at room temperature until the reaction was complete. The product was crystallized by the addition of 2-methyl-THF. The suspension was stirred at room temperature. The solid was isolated by filtration, washed with methyl-THF and dried under vacuum. 202 mg of white solid was obtained; (yield: 64, 56%).

LiBr (0.036 g, 2 eq) was added to a suspension of tiotropium triflate (0.114 g, 0.21 mmol) in MeCN (10 mL) at r.t. The reaction mixture was stirred for 1 h30. The solid formed was isolated by filtration and washed with dichloromethane to afford 0.077 g (78%) of tiotropium bromide as a white solid; Mp=235° C. ¹H NMR (DMSO-d₆, 400 MHz): d 7.54-7.52 (2H, m), 7.41 (1H, s), 7.15-7.13 (2H, m), 7.03-7.01 (2H, m), 5.14 (1H, t, J=5.9 Hz), 4.16 (2H, d, J=2.8 Hz), 3.52 (2H, s), 3.34. (1H, s), 3.27 (3H, s), 3.07 (3H, s), 2.72-2.65 (2H, m), 1.91 (2H, d, J=17.3 Hz). ¹³C NMR (DMSO-d₆, 100 MHz): d 169.7, 146.6, 126.7, 126.1, 125.7, 76.2, 64.4, 63.6, 55.5, 53.6, 47.1, 28.2. FT-IR (KBr): 3139, 3095, 3054, 1729, 1664, 1635, 1434, 1411, 1355, 1336, 1321, 1253, 1224, 1203, 1172, 1159, 1068, 1037 cm⁻¹.

### Example 12

### Preparation of (10R,13S,17R)-methyl 11,17-dihydroxy-10,13-dimethyl-3-oxo-6,7,8,9,10,11,12,13,14,15,16,17-dodecahydro-3H-cyclopenta[a]phenanthrene-17-carboxylate

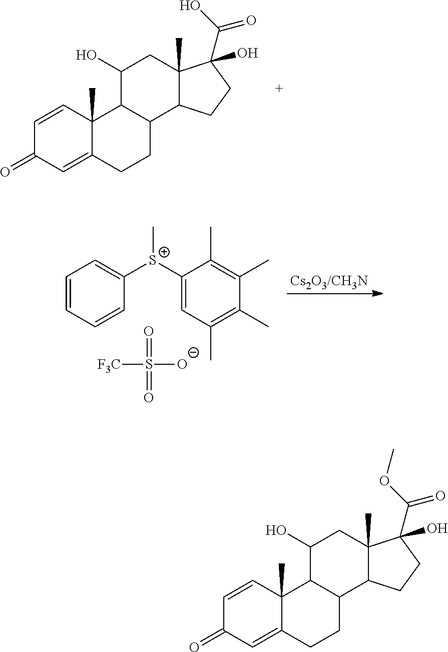 [SMILES: C[C@]12C=CC(=O)C=C1CCC1C2C(O)C[C@@]2(C)C1CC[C@@]2(O)C(=O)O ; Cc1cc([S+](C)c2ccccc2)c(C)c(C)c1C ; O=S(=O)([O-])C(F)(F)F ; COC(=O)[C@]1(O)CCC2C3CCC4=CC(=O)C=C[C@]4(C)C3C(O)C[C@@]21C]

(10R,13S,17R)-11,17-dihydroxy-10,13-dimethyl-3-oxo-6,7,8,9,10,11,12,13,14,15,16,17-dodecahydro-3H-cyclopenta[a]phenanthrene-17-carboxylic acid (0.5 g, 1.44 mmol) was suspended in dichloromethane (5 ml). Cesium carbonate (587 mg, 1.0 eq) and methyl (phenyl) (2,3,4,5-tetramethylphenyl) sulfonium trifluoromethanesulfonate (587 mg, 1 eq) of example 2, were added. The resulting suspension was stirred at room temperature until the reaction was complete. Heptane (15 mL) was added and the suspension was stirred for 2 hours. The solid was isolated by filtration and dried under vacuum at a temperature below 50° C. The solid was recrystallized from a mixture of acetone and water. The salts are purged during this recrystallization.

It is evident to one skilled in the art that this invention is not limited to the foregoing examples, and that can be embodied in other specific forms without departing from the scope of the invention. Thus, the examples should be considered as illustrative and not restrictive, reference being made to the claims, and that all changes which come within the meaning and range of equivalency of claims be embraced therein.

1-18. (canceled)

19. A compound of Formula II, or a salt thereof, 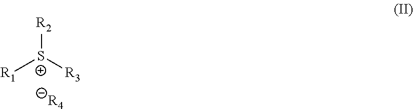
wherein,
R₁ is an aryl group;
R₂ is an alkyl group;
R₃ is an unsubstituted or alkyl-substituted phenyl group, wherein the number of alkyl substituents (n) is greater than 2; and
R₄ is an anion.

20. The compound according to claim 19, wherein R₁ is a C₆₋₁₂ aryl group.

21. The compound according to claim 19 wherein R₁ is a phenyl or p-tolyl group.

22. The compound according to claim 20 wherein R₁ is a phenyl or p-tolyl group.

23. The compound according to claim 19, wherein R₂ is a linear or branched C₁₋₈ alkyl group.

24. The compound according to claim 19 wherein R₂ is selected from the group consisting of methyl, ethyl, n-propyl, iso-propyl, n-butyl, sec-butyl, iso-butyl and tert-butyl.

25. The compound according to claim 23, wherein R₂ is selected from the group consisting of methyl, ethyl, n-propyl, iso-propyl, n-butyl, sec-butyl, iso-butyl and tert-butyl.

26. The compound according to claim 19 wherein R₂ is methyl or ethyl.

27. The compound according to claim 23 wherein R₂ is methyl or ethyl.

28. The compound according to claim 19, wherein the number of alkyl substituents (n) in the phenyl group is 3, 4 or 5.

29. The compound according to claim 19, wherein the number of alkyl substituents (n) in the phenyl group is 4.

30. The compound according to claim 19, wherein R₄ is a tetrafluoroborate, triflate, sulphate, phosphate or carbonate anion.

31. The compound according to claim 19, selected from the group consisting of:
S-methyl-S-phenyl-2,3,4,5-tetramethyl phenyl sulfonium triflate;
S-methyl-S-phenyl-2,3,4,5-tetramethyl phenyl sulfonium tetrafluoroborate;
S-methyl-S-phenyl-2,3,4,5-tetramethyl phenyl sulfonium hexafluorophosphate;
S-methyl-S-phenyl-2,3,4,5-tetramethyl phenyl sulfonium hexafluorophosphate;
methyl (2,3,4,5-tetramethylphenyl)(p-tolyl) sulfonium triflate;
methyl (2,3,4,5-tetramethylphenyl)(p-tolyl) sulfonium tetrafluoroborate;
S-ethyl-S-(4-methyl) phenyl-2,3,4,5-tetramethyl phenyl sulfonium triflate;
S-ethyl-S-phenyl-2,3,4,5-tetramethyl phenyl sulfonium tetrafluoroborate; and
S-ethyl-S-phenyl-2,3,4,5-tetramethyl phenyl sulfonium hexafluorophosphate.

32. A process for preparing a compound of Formula II, 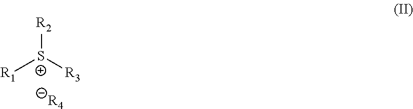
or a salt thereof, wherein,
R₁ is an aryl group;
R₂ is an alkyl group;
R₃ is an alkyl-substituted phenyl group, wherein the number of alkyl substituents (n) is greater than 2; and
R₄ is an anion
comprising
(i) reacting a compound of Formula IV,
R₁—S(═O)—R₂ (IV)
with a substituted phenyl derivative; and
(ii) reacting a compound so formed with a suitable anion source.

33. The process of claim 32 for preparing a compound of Formula II or a salt thereof, comprising first preparing a compound of Formula V, VI or VII, 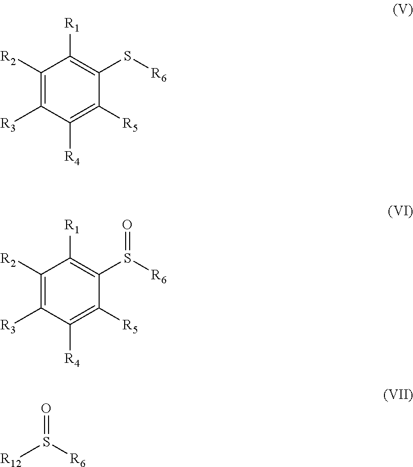
wherein,
R₁, R₂, R₃, R₄ and R₅, which may be the same or different, are selected from H, alkyl, aryl, alkoxy and halogen;
R₆ is an alkyl group; and
R₁₂ is H, substituted naphthalene or a polymer resin.

34. A compound comprising Formula III, 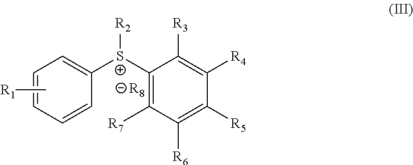
or a salt thereof
wherein,
R₁ is an alkyl group or H;
R₂ is an alkyl group;
one or two of R₃, R₄, R₅, R₆ and R₇ are H and each of the remaining R₃, R₄, R₅, R₆ and R₇, which may be the same or different, is an alkyl group; and
R₈ is an anion, chosen from the group consisting of tetrafluoroborate, triflate, sulfate, phosphate and carbonate.

35. A method of manufacture an organic biologically active compound comprising at least one alkylation step, wherein an alkylating reagent comprises Formula II, or a salt thereof, wherein Formula II is 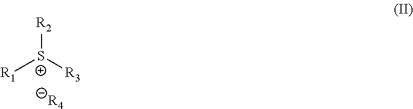
and
R₁ is an aryl group;
R₂ is an alkyl group;
R₃ is an unsubstituted or alkyl-substituted phenyl group, wherein the number of alkyl substituents (n) is greater than 2; and
R₄ is an anion.

36. The method according to claim 35, wherein the organic biologically active compound is selected from the group consisting of: demecarium bromide, fenpiverinium bromide, fentonium bromide, heteronium bromide, mepenzolate bromide, tropenziline bromide, vecuronium bromide, propantheline bromide, timepedium bromide, pancuronium bromide, penthienate bromide, pipecuronium bromide, pipenzolate bromide, tiotropium bromide, anisotropine methylbromide, hexafluorenium bromide, ipratropium bromide, 8-p-phenylbenzyltropinium bromide, oxitefonium bromide and (10R,13S,17R)-methyl 11,17-dihydroxy-10,13-dimethyl-3-oxo-6,7,8,9,10,11,12,13,14,15,16,17-dodecahydro-3H-cyclopenta-[a]phenanthrene-17-carboxylate.

37. The method according to claim 35, wherein the organic biologically active compound is tiotropium bromide, ipratropium bromide or (10R,13S,17R)-methyl 11,17-dihydroxy-10,13-dimethyl-3-oxo-6,7,8,9,10,11,12,13,14,15,16,17-dodeca-hydro-3H-cyclopenta[a]phenanthrene-17-carboxylate.

38. Use as an alkylating agent of a compound of Formula II, or a salt thereof, 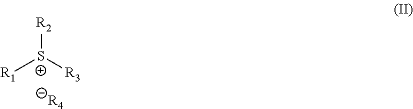
wherein,
R₁ is an aryl group;
R₂ is an alkyl group;
R₃ is an unsubstituted or alkyl-substituted phenyl group, wherein the number of alkyl substituents (n) is greater than 2; and
R₄ is an anion.

39. The use of the alkylating agent as set forth in claim 38 to produce an organic biologically active compound selected from the group consisting of: demecarium bromide, fenpiverinium bromide, fentonium bromide, heteronium bromide, mepenzolate bromide, tropenziline bromide, vecuronium bromide, propantheline bromide, timepedium bromide, pancuronium bromide, penthienate bromide, pipecuronium bromide, pipenzolate bromide, tiotropium bromide, anisotropine methylbromide, hexafluorenium bromide, ipratropium bromide, 8-p-phenylbenzyltropinium bromide, oxitefonium bromide and (10R,13S,17R)-methyl 11,17-dihydroxy-10,13-dimethyl-3-oxo-6,7,8,9,10,11,12,13,14,15,16,17-dodecahydro-3H-cyclopenta-[a]phenanthrene-17-carboxylate.

40. The use of the alkylating agent as set forth in claim 38 to produce an organic biologically active compound selected from the group consisting of tiotropium bromide, ipratropium bromide or (10R,13S,17R)-methyl 11,17-dihydroxy-10,13-dimethyl-3-oxo-6,7,8,9,10,11,12,13,14,15,16,17-dodeca-hydro-3H-cyclopenta[a]phenanthrene-17-carboxylate.
